(19) United States
(12) Patent Application Publication
(10) Pub. No.: US 2010/0152200 A1
(43) Pub. Date: Jun. 17, 2010

(54) ALPHAVIRUS INHIBITORS AND USES THEREOF

(75) Inventors: David J. Miller, Chelsea, MI (US); Daniel C. Peltier, Ann Arbor, MI (US); Weiping Peng, Ann Arbor, MI (US)

Correspondence Address: Casimir Jones, S.C., 2275 DEMING WAY, SUITE 310, MIDDLETON, WI, 53562, US

(73) Assignee: THE REGENTS OF THE UNIVERSITY OF MICHIGAN, Ann Arbor, MI (US)

(21) Appl. No.: 12/619,117

(22) Filed: Nov. 16, 2009

Related U.S. Application Data
(60) Provisional application No. 61/115,312, filed on Nov. 17, 2008.

(51) Int. Cl. A61K 31/496; C07D 495/04; A61K 31/454; C07D 401/14; A61P 31/12

(52) U.S. Cl. 514/253.09; 546/198; 514/321; 544/364

(57) ABSTRACT

The present invention relates to chemical compounds, methods for their discovery, and their therapeutic use. In particular, the present invention provides compounds as inhibitors of alphaviruses.

### CROSS REFERENCE TO RELATED APPLICATIONS

This application claims the benefit of U.S. Provisional Application Ser. No. 61/115,312, filed Nov. 17, 2008, which is herein incorporated by reference in its entirety.

### GOVERNMENT SUPPORT

This application was made with government support under grant number U54 AI057153 awarded by the National Cancer Institute. The government has certain rights in the invention.

### FIELD OF THE INVENTION

The present invention relates to chemical compounds, methods for their discovery, and their therapeutic use. In particular, the present invention provides compounds as inhibitors of alphaviruses.

### BACKGROUND OF THE INVENTION

The Alphavirus genus within the Togaviridae family contains about 30 mosquito-borne, enveloped, positive-stranded RNA viruses, one third of which cause significant diseases in human and animals worldwide. For example, neurotropic alphaviruses such as western, eastern, and Venezuelan equine encephalitis viruses (WEEV, EEEV, and VEEV, respectively) infect the central nervous system (CNS) and can lead to severe encephalitis in humans with fatality rates of up to 70%, and where survivors often bear long-term neurological sequelae (Deresiewicz et al., N Engl J Med 1997; 336:1867-74; Earnest et al., Neurology 1971; 21:969-74). Neurotropic alphaviruses are also important members of the growing list of emerging or resurging public health threats (Gubler, Arch Med Res 2002; 33:330-42) and are listed as CDC and NIAID category B bioterrorism agents, due in part to numerous characteristics that make them potential biological weapons: (i) high clinical morbidity and mortality; (ii) potential for aerosol transmission; (iii) lack of effective countermeasures for disease prevention or control; (iv) public anxiety elicited by CNS infections; (v) ease with which large volumes of infectious materials can be produced; and (vi) potential for malicious introduction of foreign genes designed to increase alphavirus virulence (Sidwell et al., Antiviral Res 2003; 57:101-11).

There are currently no licensed vaccines or antiviral drugs for alphaviruses. Formalin inactivated vaccines for WEEV or EEEV and a live attenuated VEEV vaccine (TC-83 strain) are available on an investigational drug basis, and are limited primarily to laboratory workers conducting research on these viruses (Sidwell et al., supra). However, these vaccines have poor immunogenicity, require annual boosters, and have a high frequency of adverse reactions. The development of alternative live attenuated, chimeric, and DNA-based alphavirus vaccines is being actively pursued, and several candidates have been tested in animal models (Barabe et al., Vaccine 2007; 25:6271-6; Wu et al., Vaccine 2007; 25:4368-75; Nagata et al., Vaccine 2005; 23:2280-3; Schoepp et al., Virology 2002; 302:299-309; Turell et al., Am J Trop Med Hyg 1999; 60:1041-4; Wang et al., Vaccine 2007; 25:7573-81; Fine et al., Vaccine 2008; 26:3497-506; Turell et al., Am J Trop Med Hyg 2008; 78:328-32). Nevertheless, the broad clinical application of these newer generation vaccines is likely years away. Furthermore, combined active vaccination and antiviral therapy may be a more effective response to an outbreak due to either natural transmission or intentional exposure to a viral pathogen (Bronze et al., Curr Opin Investig Drugs 2003; 4:172-8).

Several compounds have been reported to inhibit alphavirus replication, including the nucleoside analogs ribavirin and mycophenolic acid (Malinoski et al., Virology 1981; 110:281-9), (−)-carbodine, triaryl pyrazoline (Puig-Basagoiti et al., Antimicrob Agents Chemother 2006; 50:1320-9), and seco-pregnane steroids from the Chinese herbs Strobilanthes cusia and Cynanchum paniculatum (Li et al., Proc Natl Acad Sci USA 2007; 104:8083-8).

Nevertheless, there is still a pressing need to identify new antiviral drugs to treat these virulent pathogens.

### SUMMARY

The present invention relates to chemical compounds, methods for their discovery, and their therapeutic use. In particular, the present invention provides compounds as inhibitors of alphaviruses.

For example, in some embodiments, the present invention provides a composition, comprising a compound of the formula:

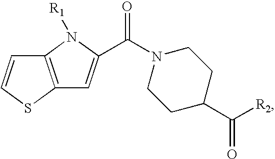

where R₁ and R₂ are the same or different and selected from, for example, an aliphatic group, a substituted aliphatic group, a cycloaliphatic group, a substituted cycloaliphatic group, a heterocyclic group, an aryl group, a substituted aryl group, or a halogen. In some embodiments, R₁ is —CH₃, —CH₂CH₃,

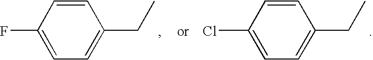

In some embodiments, R₂ is

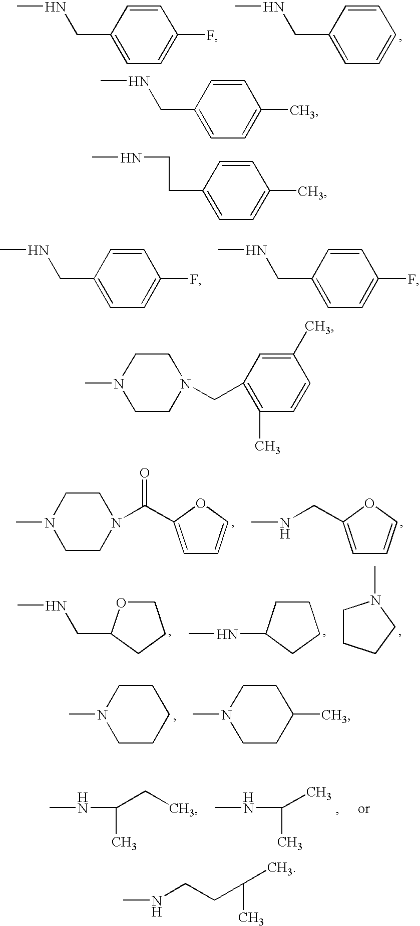

In some embodiments, the compound is

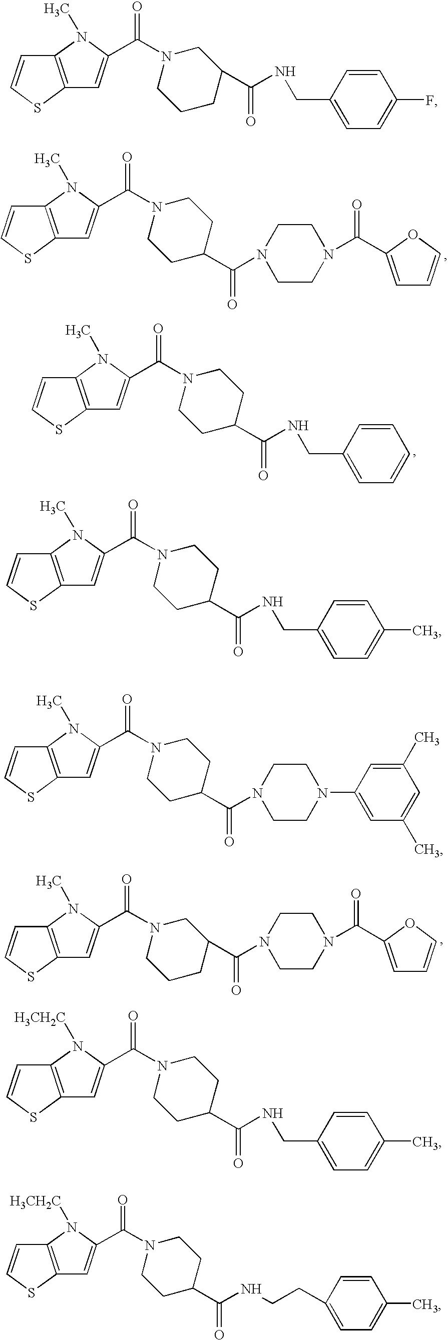

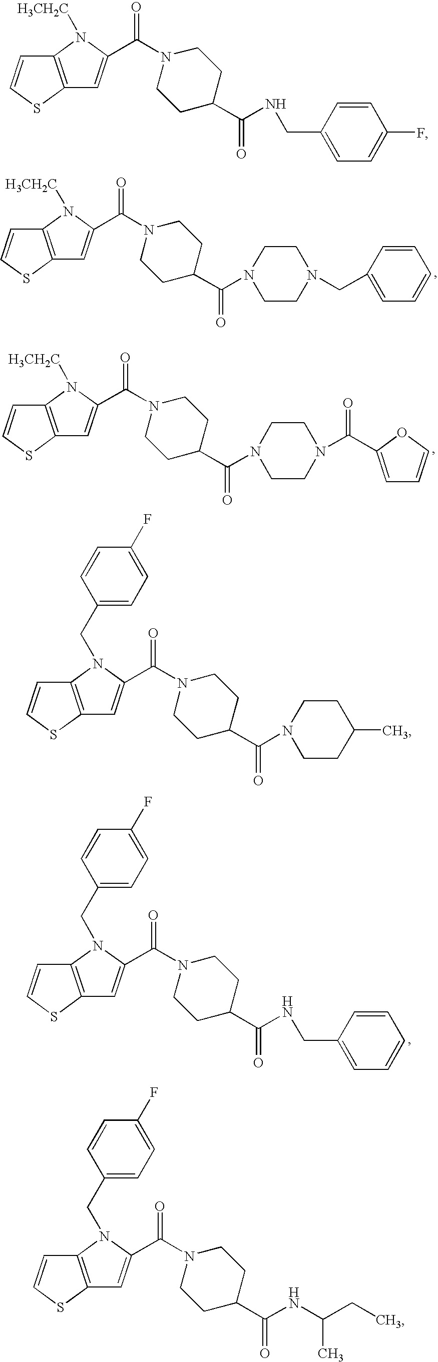

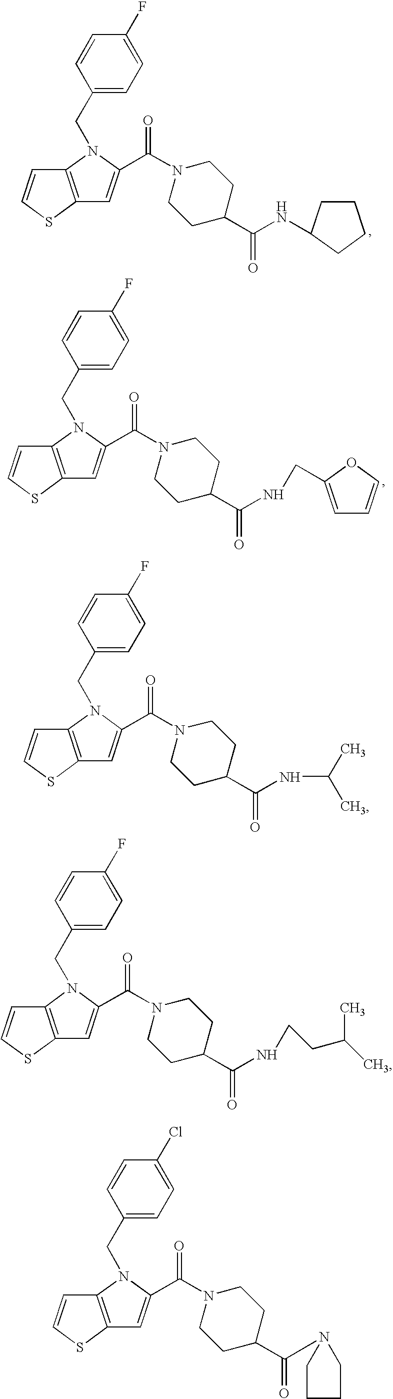

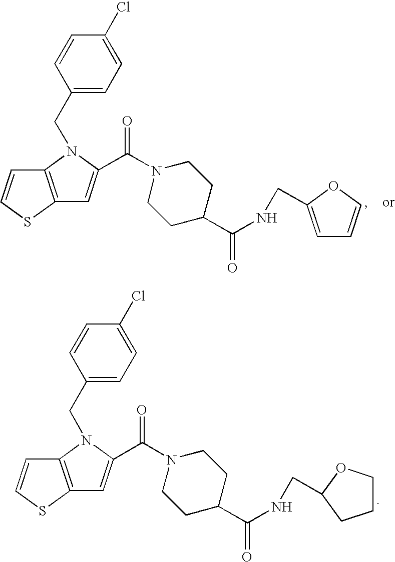

In some embodiments, the compound is

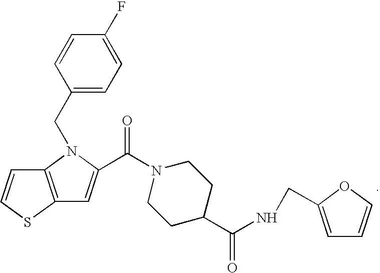

Embodiments of the present invention further provide derivatives, mimetics, stereoisomers, salts, etc. of the above named compounds.

Embodiments of the present invention further provide pharmaceutical preparations comprising the aforementioned compounds and a pharmaceutically acceptable carrier.

Additional embodiments of the present invention provide methods of using the aforementioned compounds to kill or inhibit the growth of an alphavirus (e.g., Sindbis virus, Semliki forest virus, O'nyong'nyong virus, Chikungunya virus, Mayaro virus, Ross River virus, Barmah Forest virus, Eastern equine encephalitis virus, Western equine encephalitis virus, or Venezuelan equine encephalitis virus). In some embodiments, the alphavirus is in a cell. In some embodiments, the cell is in an animal. In some embodiments, the animal exhibits symptoms of an alphavirus infection and contacting the cell with the compound results in a decrease or elimination of symptoms of an alphavirus infection.

### BRIEF DESCRIPTION OF THE DRAWINGS

FIG. 1 shows cell-based WEEV replicon system for HTS. (A) Schematic of WEEV replicon pWR-LUC. Region delete for the control plasmid pWR-ΔLUC is indicated by the dashed lines. U, untranslated region; An, polyadenylated tail. (B) fLUC reporter gene activity in BSR-T7/5 cells transfected with empty vector, pWR-LUC, or control pWR-ΔLUC. Results are expressed as relative luciferase units (RLU). (C) BSR-T7/5 cells transfected with pWR-LUC were treated with no inhibitor, 50 μM ribavirin (Rib), or 5 μM mycophenolic acid (MPA), and fLUC activity was measured after 18 h. Results are expressed as percentage of fLUC activity relative to untreated control.

FIG. 2 shows that CCG32091 potently inhibits WEEV replicon activity with minimal cytotoxicity. (A) Dose-response curves of BSR-T7/5 cells transfected with pWR-LUC and treated with increasing concentrations of CCG32091. (B) Structure of CCG32091. The R1 and R2 groups central to the SAR (see table 2) are highlighted by boxes.

FIG. 3 shows that CCG32091 inhibits alphavirus replication in cultured human neuronal cells. (A) Human BE(2)-C neuronal cells were infected with FMV (black bars) or SINV (white bars) at an MOI of 0.1 and simultaneously treated with no inhibitor, 12.5 μM CCG32091, or 50 μM ribavirin (Rib), and cell viability was determined at 48 h after infection by MTT assay. (B) BE(2)-C cells were infected with FMV at an MOI of 1, treated with inhibitors as described above, and virus titers in culture supernatants were determined at 24 h after infection by plaque assay. (C) BE(2)-C cells were infected with WEEV at an MOI of 1, treated with CCG32091, and viral RNA corresponding to nsP2 and E1 regions were analyzed by RT-PCR at 6 h after infection. (D) BE(2)-C cells were infected with FMV (black bars) or WEEV (grey bars) and treated with CCG32091 as described above, and viral RNA levels corresponding to the E1 gene were determined by quantitative RT-PCR.

### DEFINITIONS

To facilitate an understanding of the present invention, a number of terms and phrases are defined below.

As used herein, the term “aliphatic” represents the groups including, but not limited to, alkyl, alkenyl, alkynyl, alicyclic.

As used herein, the term “aryl” represents a single aromatic ring such as a phenyl ring, or two or more aromatic rings (e.g., bisphenyl, naphthalene, anthracene), or an aromatic ring and one or more non-aromatic rings. The aryl group can be optionally substituted with a lower aliphatic group (e.g., alkyl, alkenyl, alkynyl, or alicyclic). Additionally, the aliphatic and aryl groups can be further substituted by one or more functional groups including, but not limited to, chemical moieties comprising N, S, O, —NH₂, —NHCOCH₃, —OH, lower alkoxy (C₁-C₄), and halo (—F, —Br, or —I).

As used herein, the term “substituted aliphatic” refers to an alkane possessing less than 10 carbons where at least one of the aliphatic hydrogen atoms has been replaced by a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such include, but are not limited to, 1-chloroethyl and the like.

As used herein, the term “substituted aryl” refers to an aromatic ring or fused aromatic ring system consisting of no more than three fused rings at least one of which is aromatic, and where at least one of the hydrogen atoms on a ring carbon has been replaced by a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, hydroxyphenyl and the like.

As used herein, the term “cycloaliphatic” refers to a cycloalkane possessing less than 8 carbons or a fused ring system consisting of no more than three fused cycloaliphatic rings. Examples of such include, but are not limited to, decalin and the like.

As used herein, the term “substituted cycloaliphatic” refers to a cycloalkane possessing less than 10 carbons or a fused ring system consisting of no more than three fused rings, and where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, 1-chlorodecalyl, bicyclo-heptanes, octanes, and nonanes (e.g., nonrbornyl) and the like.

As used herein, the term “heterocyclic” refers to a cycloalkane and/or an aryl ring system, possessing less than 8 carbons, or a fused ring system consisting of no more than three fused rings, where at least one of the ring carbon atoms is replaced by oxygen, nitrogen or sulfur. Examples of such include, but are not limited to, morpholino and the like.

As used herein, the term “substituted heterocyclic” refers to a cycloalkane and/or an aryl ring system, possessing less than 8 carbons, or a fused ring system consisting of no more than three fused rings, where at least one of the ring carbon atoms is replaced by oxygen, nitrogen or sulfur, and where at least one of the aliphatic hydrogen atoms has been replaced by a halogen, hydroxy, a thio, nitro, an amino, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to 2-chloropyranyl.

As used herein, the term “lower-alkyl-substituted-amino” refers to any alkyl unit containing up to and including eight carbon atoms where one of the aliphatic hydrogen atoms is replaced by an amino group. Examples of such include, but are not limited to, ethylamino and the like.

As used herein, the term “lower-alkyl-substituted-halogen” refers to any alkyl chain containing up to and including eight carbon atoms where one of the aliphatic hydrogen atoms is replaced by a halogen. Examples of such include, but are not limited to, chlorethyl and the like.

The term “derivative” of a compound, as used herein, refers to a chemically modified compound wherein the chemical modification takes place at any location of the compound (e.g., at a functional group).

As used herein, the term “subject” refers to organisms to be treated by the methods of the present invention. Such organisms preferably include, but are not limited to, mammals (e.g., murines, simians, equines, bovines, porcines, canines, felines, and the like), and most preferably includes humans. In the context of the invention, the term “subject” generally refers to an individual who will receive or who has received treatment (e.g., administration of a compound of the present invention and optionally one or more other agents) for a condition characterized by infection by alphavirus or risk of infection by alphavirus.

The term “diagnosed,” as used herein, refers to the recognition of a disease by its signs and symptoms (e.g., resistance to conventional therapies), or genetic analysis, pathological analysis, histological analysis, diagnostic assay (e.g., for alphavirus infection) and the like.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the term “host cell” refers to any eukaryotic or prokaryotic cell (e.g., mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route.

As used herein, the term “co-administration” refers to the administration of at least two agent(s) (e.g., a compound of the present invention) or therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “toxic” refers to any detrimental or harmful effects on a cell or tissue as compared to the same cell or tissue prior to the administration of the toxicant.

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “sample” as used herein is used in its broadest sense. A sample suspected of indicating the presence of an alphavirus may comprise a cell, tissue, or fluids, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the terms “purified” or “to purify” refer, to the removal of undesired components from a sample. As used herein, the term “substantially purified” refers to molecules that are at least 60% free, preferably 75% free, and most preferably 90%, or more, free from other components with which they usually associated.

As used herein, the term “modulate” refers to the activity of a compound (e.g., a compound of the present invention) to affect (e.g., to kill or prevent the growth of) an alphavirus.

The term “test compound” refers to any chemical entity, pharmaceutical, drug, and the like, that can be used to treat or prevent a disease, illness, sickness, or disorder of bodily function, or otherwise alter the physiological or cellular status of a sample (e.g., infection by alphavirus). Test compounds comprise both known and potential therapeutic compounds. A test compound can be determined to be therapeutic by using the screening methods of the present invention. A “known therapeutic compound” refers to a therapeutic compound that has been shown (e.g., through animal trials or prior experience with administration to humans) to be effective in such treatment or prevention. In some embodiments, “test compounds” are agents that treat or prevent alphavirus infection.

### DETAILED DESCRIPTION OF THE INVENTION

The present invention relates to chemical compounds, methods for their discovery, and their therapeutic use. In particular, the present invention provides compounds as inhibitors of alphaviruses.

The neurotropic alphaviruses represent emerging pathogens with the potential for widespread dissemination and the ability to cause substantial morbidity and mortality (Gubler, Arch Med Res 2002; 33:330-42; Sidwell et al., Antiviral Res 2003; 57:101-11), but for which no licensed therapies currently exist. Experiments conducted during the course of development of embodiments of the present invention resulted in the identification of thieno[3,2-b]pyrrole compounds with inhibitory activity against alphaviruses. Heterocyclic compounds that contain a thieno[3,2-b]pyrrole core have been previously identified as possessing physiological activity with potential clinical applications, including uses as anti-inflammatory agents (Kumar et al., Bioorg Med Chem 2004; 12:1221-30), glycogen phosphorylase inhibitors for diabetes treatment (Whittamore et al., Bioorg Med Chem Lett 2006; 16:5567-71), and hepatitis C virus (HCV) inhibitors (Ontoria et al., Bioorg Med Chem Lett 5 2006; 16:4026-30).

Alphaviruses, like all other group IV viruses, have a positive sense single stranded RNA genome. There are 27 alphaviruses, able to infect various vertebrates such as humans, rodents, birds, and larger mammals such as horses as well as invertebrates. Transmission between species and individuals occurs via mosquitoes, making the alphaviruses a contributor to the collection of Arboviruses—or Arthropod Borne Viruses. Alphaviruses particles are enveloped, have a 70 nm diameter, tend to be spherical (although slightly pleomorphic), and have a 40 nm isometric nucleocapsid. Table 3 shows medically important Alphaviruses and details of their human disease, vertebrate reservoir and distribution.

TABLE 3
Medically Important Alphaviruses

| | | Vertebrate | |
|---|---|---|---|
| Virus | Human Disease | Reservoir | Distribution |
| Sindbis virus | Rash, arthritis | Birds | Europe, Africa, |
| | | | Australia |
| Semliki forest virus | Rash | Birds | Africa |
| O'nyong'nyong virus | Rash | Primates | Africa |
| Chikungunya virus | Rash | Primates, | Africa, India, |
| | | humans | SE Asia |
| Mayaro virus | Rash | Primates, | South America |
| | | humans | |
| Ross River virus | Rash | Mammals, | Australia, |
| | | humans | South Pacific |
| Barmah Forest virus | Fever, malaise, | Humans | Australia |
| | rash, joint pain, | | |
| | muscle tenderness | | |
| Eastern equine | Encephalitis | Birds | Americas |
| encephalitis virus | | | |
| Western equine | Encephalitis | Birds, | North America |
| encephalitis virus | | mammals | |
| Venezuelan equine | Encephalitis | Rodents, | Americas |
| encephalitis virus | | horses | |

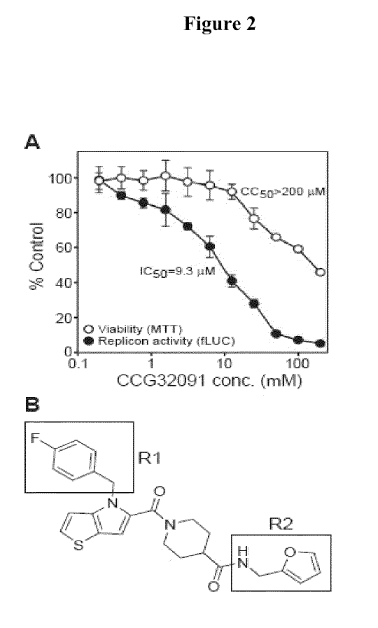

One compound identified in experiments described herein, CCG32091 (FIG. 2), is a PubChem registered compound (CID: 3240671) and part of the NIH Molecular Libraries-Small Molecule Repository (MLSMR), and has been identified as an active compound in only 5 of ˜250 HTS assays conducted through the NIH Molecular Libraries Screening Center Network (MLSCN). This indicates that the spectrum of its biological activity is fairly narrow, which is a highly desirable attribute in a potential lead compound. In addition, Ilyin et al. recently described a solution-phase strategy for the synthesis of novel combinatorial libraries containing a thieno[3,2-b]pyrrole core (Ilyin et al., J Comb Chem 2007; 9:96-106).

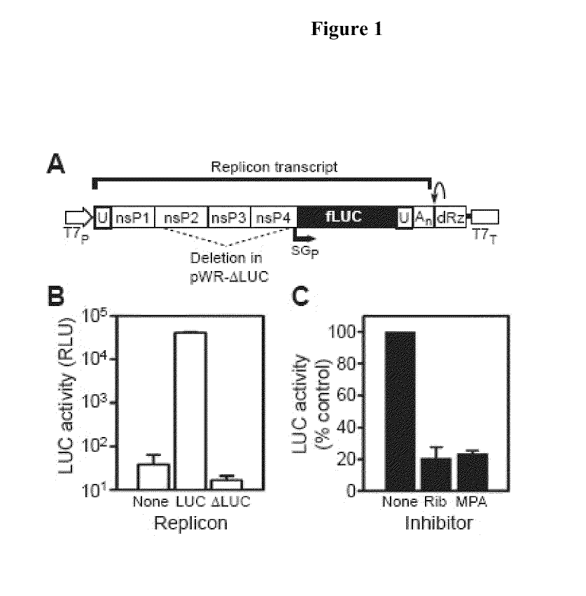

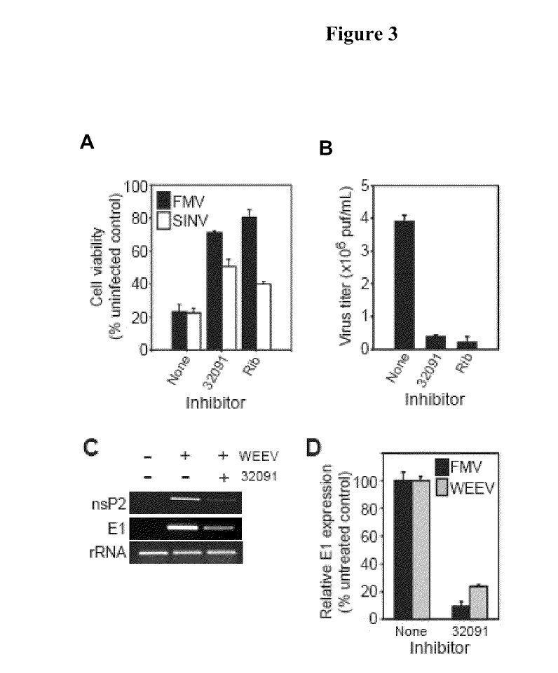

The mechanism(s) underlying the antiviral activity of thieno[3,2-b]pyrroles against neurotropic alphaviruses is unknown. The present invention is not limited to a particular mechanism. Indeed, an understanding of the mechanism is not necessary to practice the present invention. Nonetheless, it is contemplated that the use of a replicon-based assay for the HTS and validation steps (FIG. 1 and Table 1) implicates viral replicase proteins as potential targets. This hypothesis is supported by the observation that CCG32091 reduced viral RNA accumulation after infection of neuronal cells (FIG. 3). Furthermore, the broad activity of CCG32091 against infectious virus or replicons derived from WEEV, EEEV, VEEV, FMV, and SINV indicates that a highly conserved viral enzymatic activity may be targeted. Alphavirus nsPs contain several distinct enzymatic activities, including methyltransferase (nsP1) (Ahola et al., J Virol 1997; 71:392-7) protease and helicase (nsP2) (Gomez et al., FEBS Lett 1999; 448:19-22; Hardy et al., J Virol 1989; 63:4653-64), and RNA polymerase (nsP4) (Poch et al., EMBO J. 1989; 8:3867-74). In vitro assays have been developed for several of these activities (Ahola et al., supra; Vasiljeva et al., J Biol Chem 2001; 276:30786-93; Tomar et al., J Virol 2006; 80:9962-9). Certain embodiments utilize in vitro screening for target identification. An alternative approach that takes advantage of the intrinsically high error rate of viral RNA polymerases previously used successfully for antiviral target identification is the isolation and characterization of viral escape mutants (Li et al., J Virol 2004; 78:9645-51; Lin et al., Virology 2000; 272:61-71; Scheidel et al., Virology 1991; 181:490-9).

The treatment of CNS infections presents an additional hurdle to overcome, as the blood-brain-barrier (BBB) represents a formidable obstacle for drug penetration (Pardridge, NeuroRx 2005; 2:1-2). The BBB is a highly effective physiologic barrier whose primary function is to closely regulate access of blood stream components to the CNS. Although infectious and inflammatory CNS diseases often disrupt BBB function and increase permeability, drug penetration remains an important aspect to consider in the development of antiviral agents against neurotropic alphaviruses. Multiple physical and chemical factors influence CNS penetration of drugs, including lipophilicity, ionization properties, molecular flexibility, polar surface area (PSA), and size (Pajouhesh et al., NeuroRx 2005; 2:541-53). The latter two properties are particularly important, where studies of marketed CNS and non-CNS drugs indicate that PSA 20 values less than 60-90 Å2 and MW less than 450 Da are required for adequate penetration (Kelder et al., Pharm Res 1999; 16:1514-9; van de Waterbeemd et al., J Drug Target 1998; 6:151-65). The thieno[3,2-b]pyrrole compound CCG32091 (FIG. 2B), has a calculated PSA of 67.5 Å2 and MW of 466 Da (PubChem database). Several of the compounds identified in the SAR (Table 2) had lower PSAs and MWs than CCG32091.

### I. Alphavirus Inhibitors

As described herein, embodiments of the present invention provide thieno[3,2-b]pyrrole based compounds for use in inhibiting the alphavirus replication, infectivity or ability to cause disease. In some embodiments, the compositions of the present invention have the structure:

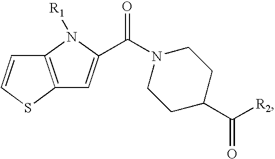

where R₁ and R₂ are the same or different and selected from, for example, any aliphatic, substituted aliphatic, cycloaliphatic, substituted cycloaliphatic, heterocyclic, aryl, substituted aryl, or halogen. In some embodiments, the R₁ and R₂ groups listed in Table 2 are utilized. For example, in some embodiments, R₁ is —CH₃, —CH₂CH₃,

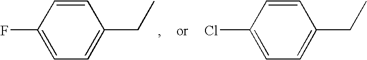

### and R₂ is

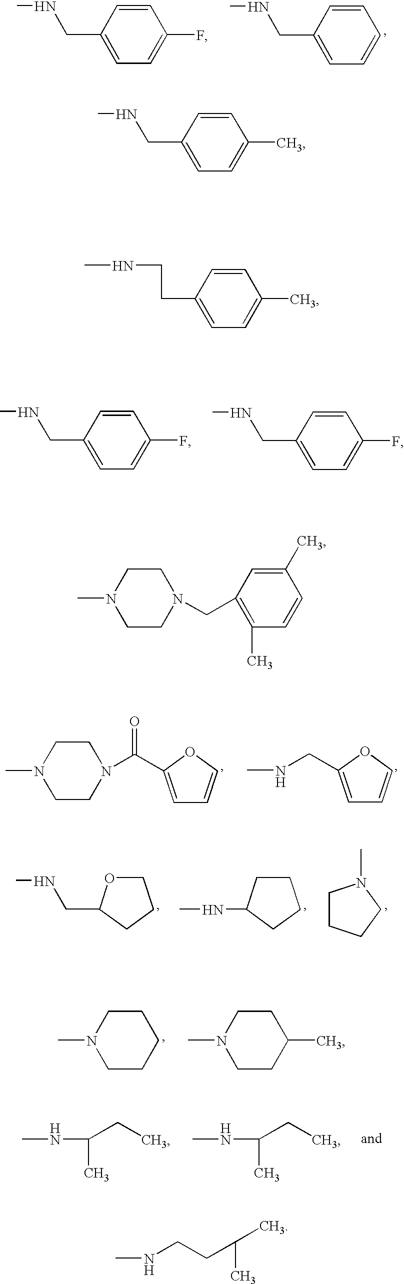

In some embodiments, the compound is

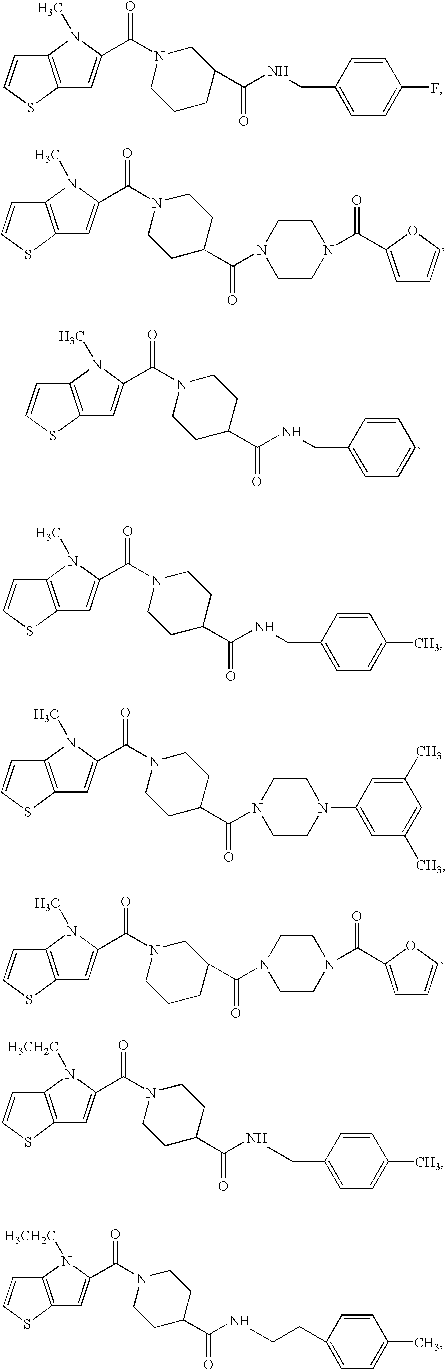

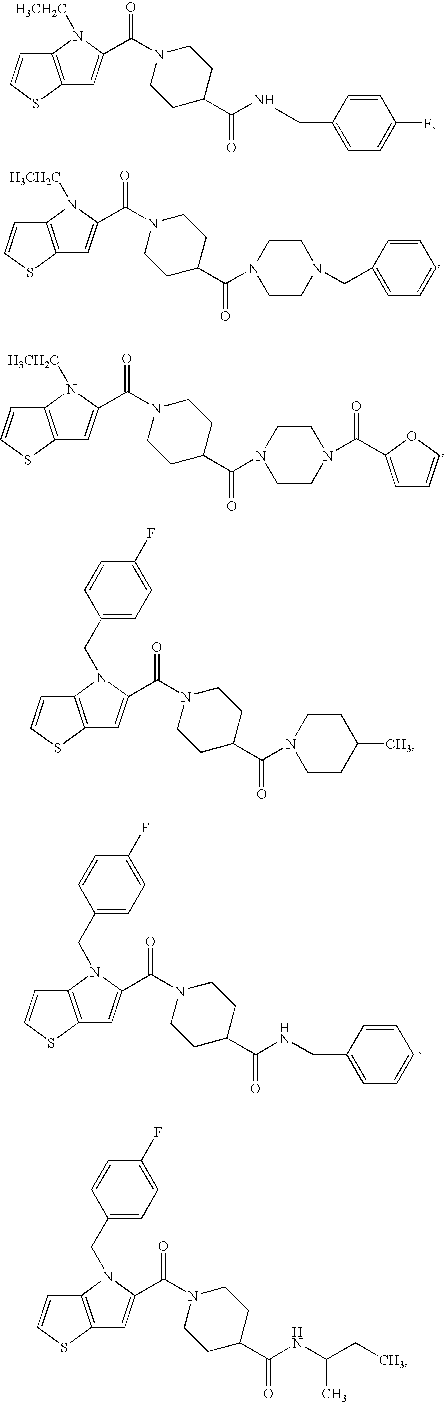

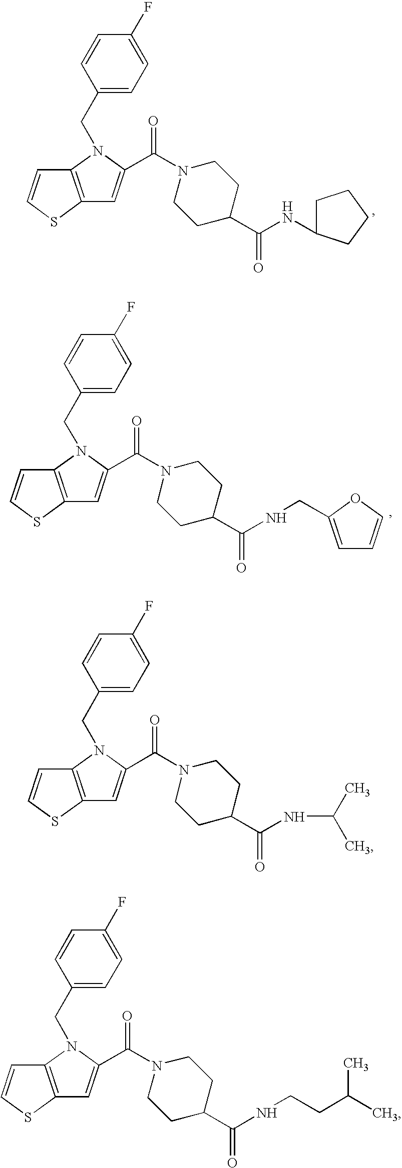

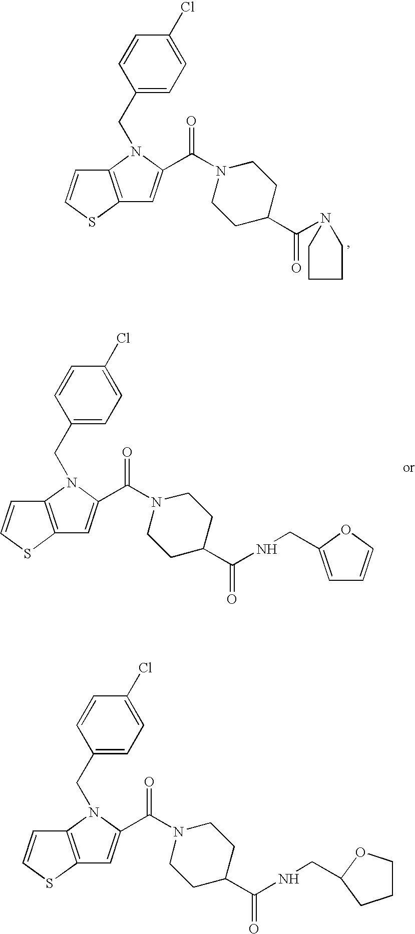

or a mimetic or derivative thereof.

The present invention also provides methods of modifying and derivatizing the compositions of the present invention to increase desirable properties (e.g., binding affinity, activity, and the like), or to minimize undesirable properties (e.g., nonspecific reactivity, toxicity, and the like). The principles of chemical derivatization are well understood. In some embodiments, iterative design and chemical synthesis approaches are used to produce a library of derivatized child compounds from a parent compound. In some embodiments, rational design methods are used to predict and model in silico ligand-receptor interactions prior to confirming results by routine experimentation.

The compounds of embodiments of the invention (or derivatives, mimetics, variants, etc. thereof) can be prepared from readily available starting materials using known methods. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds of embodiments of this invention contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

### II. Pharmaceutical Compositions, Formulations, and Exemplary Administration Routes and Dosing Considerations

Exemplary embodiments of various contemplated medicaments and pharmaceutical compositions are provided below.

A. Preparing Medicaments

The compounds of the present invention are useful in the preparation of medicaments to treat or prevent alphavirus infection. The methods and techniques for preparing medicaments of a compound are well-known in the art. Exemplary pharmaceutical formulations and routes of delivery are described below.

One of skill in the art will appreciate that any one or more of the compounds described herein, including the many specific embodiments, are prepared by applying standard pharmaceutical manufacturing procedures. Such medicaments can be delivered to the subject by using delivery methods that are well-known in the pharmaceutical arts.

B. Exemplary Pharmaceutical Compositions and Formulation

In some embodiments of the present invention, the compositions are administered alone, while in some other embodiments, the compositions are preferably present in a pharmaceutical formulation comprising at least one active ingredient/agent (e.g., alphavirus inhibitor), as defined above, together with a solid support or alternatively, together with one or more pharmaceutically acceptable carriers and optionally other therapeutic agents. Each carrier should be “acceptable” in the sense that it is compatible with the other ingredients of the formulation and not injurious to the subject.

Contemplated formulations include those suitable oral, rectal, nasal, topical (including transdermal, buccal and sublingual), vaginal, parenteral (including subcutaneous, intramuscular, intravenous and intradermal) and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient; as a powder or granules; as a solution or suspension in an aqueous or non-aqueous liquid; or as an oil-in-water liquid emulsion or a water-in-oil liquid emulsion. In some embodiments, the active ingredient is presented as a bolus, electuary, or paste, etc.

In some embodiments, tablets comprise at least one active ingredient and optionally one or more accessory agents/carriers are made by compressing or molding the respective agents. In some embodiments, compressed tablets are prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets are made by molding in a suitable machine a mixture of the powdered compound (e.g., active ingredient) moistened with an inert liquid diluent. Tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention are optionally formulated as ointments, creams, suspensions, lotions, powders, solutions, pastes, gels, sprays, aerosols or oils. In alternatively embodiments, topical formulations comprise patches or dressings such as a bandage or adhesive plasters impregnated with active ingredient(s), and optionally one or more excipients or diluents. In some embodiments, the topical formulations include a compound(s) that enhances absorption or penetration of the active agent(s) through the skin or other affected areas. Examples of such dermal penetration enhancers include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in an known manner. This phase typically comprises a lone emulsifier (otherwise known as an emulgent), it is also desirable in some embodiments for this phase to further comprises a mixture of at least one emulsifier with a fat or an oil or with both a fat and an oil.

Preferably, a hydrophilic emulsifier is included together with a lipophilic emulsifier so as to act as a stabilizer. It some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate.

Formulations suitable for vaginal administration may be presented as pessaries, creams, gels, pastes, foams or spray formulations containing in addition to the agent, such carriers as are known in the art to be appropriate.

Formulations suitable for nasal administration, wherein the carrier is a solid, include coarse powders having a particle size, for example, in the range of about 20 to about 500 microns which are administered in the manner in which snuff is taken, i.e., by rapid inhalation (e.g., forced) through the nasal passage from a container of the powder held close up to the nose. Other suitable formulations wherein the carrier is a liquid for administration include, but are not limited to, nasal sprays, drops, or aerosols by nebulizer, an include aqueous or oily solutions of the agents.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. In some embodiments, the formulations are presented/formulated in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

C. Exemplary Administration Routes and Dosing Considerations

Various delivery systems are known and can be used to administer a therapeutic agent (e.g., alphavirus inhibitor) of the present invention, e.g., encapsulation in liposomes, microparticles, microcapsules, receptor-mediated endocytosis, and the like. Methods of delivery include, but are not limited to, intra-arterial, intra-muscular, intravenous, intranasal, and oral routes. In specific embodiments, it may be desirable to administer the pharmaceutical compositions of the invention locally to the area in need of treatment; this may be achieved by, for example, and not by way of limitation, local infusion during surgery, injection, or by means of a catheter.

The agents identified herein as effective for their intended purpose can be administered to subjects or individuals susceptible to or at risk of alphavirus infection or disease. When the agent is administered to a subject such as a mouse, a rat or a human patient, the agent can be added to a pharmaceutically acceptable carrier and systemically or topically administered to the subject. To determine patients that can be beneficially treated, a tissue sample is removed from the patient and the cells are assayed for sensitivity to the agent.

In some embodiments, in vivo administration is effected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

Suitable dosage formulations and methods of administering the agents are readily determined by those of skill in the art. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including, but not limited to, oral, rectal, nasal, topical (including, but not limited to, transdermal, aerosol, buccal and sublingual), vaginal, parental (including, but not limited to, subcutaneous, intramuscular, intravenous and intradermal) and pulmonary. It is also appreciated that the preferred route varies with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient.

Desirable blood levels of the agent may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

D. Exemplary Co-Administration Routes and Dosing Considerations

The present invention also includes methods involving co-administration of the compounds described herein with one or more additional active agents. Indeed, it is a further aspect of this invention to provide methods for enhancing prior art therapies and/or pharmaceutical compositions by co-administering a compound of this invention. In co-administration procedures, the agents may be administered concurrently or sequentially. In one embodiment, the compounds described herein are administered prior to the other active agent(s). The pharmaceutical formulations and modes of administration may be any of those described above. In addition, the two or more co-administered chemical agents, biological agents or vaccines may each be administered using different modes or different formulations.

The agent or agents to be co-administered depends on the type of condition being treated. For example, when the condition being treated is alphavirus infection, the additional agent can be an antiviral agent or an agent that treats symptoms of alphavirus infection or an alphavirus vaccine. The additional agents to be co-administered can be any of the well-known agents in the art, including, but not limited to, those that are currently in clinical use. The determination of appropriate type and dosage of radiation treatment is also within the skill in the art or can be determined with relative ease.

### III. Drug Screens

In some embodiments of the present invention, the compounds of the present invention, and other potentially useful compounds, are screened for their biological activity (e.g., ability to treat or prevent alphavirus infection). In some embodiments of the present invention, the compounds of the present invention, and other potentially useful compounds, are screened for their ability to treat or prevent alphavirus infection using one of the in vitro or in vivo assays described herein.

For example, in some embodiments, drug screening applications utilize a reporter gene assay comprising alphavirus genes linked to a reporter gene to assay for alphavirus genome replication.

In some embodiments, candidate compounds identified using the reporter gene assay are further screened using cellular toxicity assays (e.g., in vitro or in vivo) or live virus assays (e.g., in vitro or in an animal model).

### IV. Therapeutic Application

In some embodiments, the present invention provides compositions and methods for treating or preventing alphavirus infection. In some embodiments, the compounds described herein (e.g., those described in Table 2) and section I above are utilized. In other embodiments, derivatives, mimetics, variants, etc. of the described compounds are utilized.

The present invention is not limited to treatment of a particular alphavirus. The compositions and methods of the present invention find use in the treatment or prevention of any number of alphaviruses, including, but not limited to, Sindbis virus, Semliki forest virus, O'nyong'nyong virus, Chikungunya virus, Mayaro virus, Ross River virus, Barmah Forest virus, Eastern equine encephalitis virus, Western equine encephalitis virus and Venezuelan equine encephalitis virus.

### EXAMPLES

The following examples are provided to demonstrate and further illustrate certain embodiments of the present invention and are not to be construed as limiting the scope thereof.

### Example 1

Methods

Cells and viruses. Human neuroblastoma (BE(2)-C), African green monkey kidney (Vero), and baby hamster kidney (BHK) cell lines were purchased from the American Type Culture Collection (ATCC, Manassas, Va.) and cultured in Dulbecco's Modified Eagle Medium containing 5% bovine grown serum (HyClone, Logan, Utah), 10 U/mL penicillin, and 10 μg/mL streptomycin. BSR-T7/5 cells, which are BHK cells that constitutively express bacteriophage T7 RNA polymerase (Buchholz et al., J Virol 1999; 73:251-9), were obtained from K. Conzelmann (Max von Pettenkofer-Institut, Munich, Germany) and were cultured in Glasgow Minimum Essential Medium containing 10% heat-inactivated fetal bovine serum, 10% tryptose phosphate broth, 1% sodium pyruvate, 0.1 mM non-essential amino acids, 10 U/mL penicillin, 10 μg/mL streptomycin, and 100-500 μg/mL G418 for selection. BHK cell lines VEErep/SEAP/Pac and EEErep/SEAP/Pac, which stably express double subgenomic VEEV or EEEV replicons with secreted alkaline phosphatase (SEAP) reporter and puromycin resistance genes (Petrakova et al., J Virol 2005; 79:7597), were obtained from I. Frolov (UTMB, Galveston, Tex.) and were cultured in Dulbecco's Modified Eagle Medium containing 7% 15 fetal bovine serum, 10 U/mL penicillin, 10 μg/mL streptomycin, and 5 μg/mL puromycin for selection. Infectious WEEV corresponding to strain Cba87 was generated as described (Castorena et al., Virology 2008; 372:208), and all experiments that involved infectious WEEV were conducted under BSL-3 conditions in approved facilities at the University of Michigan. Fort Morgan virus (FMV) strain CM4-146 was purchased from ATCC, and SINV strain Toto64 was obtained from R. Kuhn (Purdue

University, West Lafayette, Ind.). FMV and SINV stocks were prepared and quantified using Vero cells as described for WEEV (Castorena et al., supra).

WEEV replicon. The WEEV replicon plasmid pWR-LUC was generated using the full-length genomic clone pWE2000 (Nagata et al., Vaccine 2005: 23:2280). This cDNA clone contains a T7 polymerase promoter to initiate precise transcription and produce viral RNA with authentic 5′ termini. The firefly luciferase (fLUC) gene was amplified from pTRE2hyg-LUC (Clontech, Palo Alto, Calif.) by PCR without an ATG initiator codon but with engineered AvrII and BstXI sites and the resultant fragment was inserted into the AvrII-BstXI site of pWE2000. This strategy replaced the majority of the WEEV structural genes with the fLUC reporter gene, but retained the first 27 amino acids of the capsid protein to preserve the 5 predicted stem-loop region within the structural gene translation enhancer previously identified in alphaviruses (Frolov et al., J Virol 1996; 70:1182-90). pWR-LUC was further modified by placing a hepatitis δ ribozyme and T7 terminator downstream of the polyadenylation region to ensure efficient transcription termination and produce authentic viral 3′ termini (FIG. 1A). To generate the control replicon pWR-ΔLUC, the NheI-NheI fragment was deleted to remove the non-structural protein (nsP) coding region that included the majority of nsP2, 3, and 4.

Primary HTS, dose-response, and secondary validation of candidate compounds. BSR-T7/5 cells at approximately 60-70% confluence in 10 cm tissue culture plates (˜2×10⁶ cells/plate) were transfected with 15 μg pWR-LUC using 22 μl, TransIT LT-1 (Mirus, Madison, Wis.) according to the manufacturer's instructions. Six hours after transfection cells were detached with 0.05% trypsin, diluted to 6.25×10⁵ cells/mL, and 20 μL cell suspension per well was dispensed into 384-well plates preloaded with individual compounds in 30 μL media at approximately 5-10 μM. All plates contained a series of 32 wells each of negative and positive controls, which consisted of dimethyl sulfoxide and 100 μM ribavirin, respectively. Plates were cultured at 37° C. and 5% CO₂ for 18 h, 40 μL media was removed and replaced with 10 μL per well Steady-Glo luciferase reagent (Promega, Madison, Wis.), and luminescence was read on a PHERAstar multi-mode plate reader (BMG Labtech, Durham, N.C.). Individual compounds identified as primary hits as described below and in Table 1 were validated by dose-response analyses using a similar 384-well format, but with 3.3-fold serial dilutions of compounds from 100 μM to 10 nM assayed in duplicate wells. Selected compounds were purchased from the original supplier and were further analyzed by repeat dose-response and toxicity studies using a 96-well format, where cell viability was quantitated by either 3-[4,5-dimethylthizol-2-yl]-2,5-diphenyltetrazolium bromide (MTT) assay as previously described (Castorena et al., supra) or with Alamar Blue (AbD Serotec, Oxford, UK) per the manufacturer's instructions. For secondary validation VEErep/SEAP/Pac and EEErep/SEAP/Pac cell lines were cultured for 24 h with compounds but without puromycin selection and SEAP reporter gene expression was measured in supernatants using QUANTI-Blue (InvivoGen, San Diego, Calif.) per the manufacturer's instructions.

Final verification of candidate compound activity with infectious virus. Human BE(2)-C neuroblastoma cells were incubated simultaneously with candidate compounds and infectious WEEV, FMV, or SINV at a multiplicity of infection (MOI) of 1 or 0.1, and MTT viability assays, northern blots, and infectious virus quantitation by plaque assay were done at 6 to 48 h after infection as previously described (Castorena et al., supra). For RT-PCR analyses, total RNA was isolated at 6 h after infection with TRIzol reagent (Invitrogen, Carlsbad, Calif.) according to the manufacturer's instructions, digested with RQ1 DNAse (Promega), and RNA concentrations and integrity were determined by spectrophotometry and denaturing agarose gel electrophoresis. First-strand cDNA synthesis was performed with the SuperScript First-Strand Synthesis System (Invitrogen) using equal amounts of total RNA with oligo(dT) 12-16 primers. For semi-quantitative RT-PCR, 200-600 by fragments of the WEEV nsP2 and E1 genes were amplified using cDNA serial dilutions and rRNA as the loading control, and products were analyzed by agarose gel electrophoresis and ethidium bromide staining. For quantitative RT-PCR, ˜200 by 20 fragments of the WEEV or FMV E1 gene were amplified using rRNA as an internal control using iQTM SYBR Green Supermix (BioRad, Hercules, Calif.) according the manufacturer's instructions in a 96-well format with triplicate wells. Amplification and detection were done with an iCycler iQ system, and fluorescence threshold values were calculated using SDS 700 system software (Bio-Rad).

### Results

Development and validation of WEEV replicon cell-based assay for HTS. The alphavirus life cycle includes three general steps that are viable targets for antivirals: (i) attachment and entry; (ii) genome replication; and (iii) encapsidation and release. Initial efforts were focused on the second step, genome replication, in order to identify novel alphavirus inhibitors. The alphavirus genome is an 11-12 kb single-stranded positive-sense RNA molecule that is divided into two major domains (Griffin D E. Alphaviruses. In: Knipe D M, Howley P M, Griffin D E, et al., eds. Fields Virology. Fourth ed. Philadelphia: Lippincott Williams & Wilkins, 2001:917-62). The 5′ two-thirds of the alphavirus genome encodes the non-structural proteins nsP1 through nsP4, which are initially synthesized as one or two polyproteins that undergo regulated autocatalytic processing to form an active replication complex. This enzymatic complex subsequently synthesizes via a negative strand intermediate both full-length genomic RNA and a 4 kb subgenomic RNA. The latter RNA segment encodes the structural capsid protein and envelope glycoproteins, which are not required for genome replication and therefore can be readily replaced by foreign genes to produce alphavirus vectors that are self-replicating, termed replicons (Frolov et al., Proc Natl Acad Sci USA 1996; 93:11371-7).

To generate a WEEV replicon amenable to HTS, the majority of the structural genes in the full-length genomic clone pWE2000 (Schoepp et al., Virology 2002; 302:299-309) were replaced with the fLUC reporter gene (FIG. 1A). To facilitate the host cell transcription necessary to “launch” the WEEV replicon from a plasmid, a highly transfectable BHK cell line derivative, BSR-T7/5 cells, which constitutively express bacteriophage T7 RNA polymerase, was used (Buchholz et al., J Virol 1999; 73:251-9). One potential complication with using cell-based assays to identify antiviral compounds is the possibility that candidate compounds will induce type I interferon production and hence suppress virus replication indirectly. The use of BSRT7/5 cells minimizes this potential complication as BHK cells are deficient in both the production and response to type I interferons (Andzhaparidze et al., J Virol 1981; 37:1-6; Kramer et al., J Interferon Res 1983; 3:425-35; Nagai et al., J Gen Virol 1981; 55:109-16).

BSR-T7/5 cells transiently transfected with the pWR-LUC replicon produced fLUC levels approximately three logs above background (FIG. 1B). Reporter gene expression was dependent on viral RNA replication, as essentially no fLUC expression was detected in cells transfected with pWR-ΔLUC, a control plasmid in which the majority of the nsP2-4 region had been deleted (FIGS. 1A and B). Furthermore, both ribavirin and mycophenolic acid, which have previously been shown to inhibit alphavirus replication (Malinoski et al., Virology 1981; 110:281-9), suppressed fLUC expression in pWR-LUC transfected BSR-T7/5 cells by approximately 80% (FIG. 1C). It was concluded from these results that the pWR-LUC:BSR-T7/5 system functions as a convenient and robust platform to identify small molecule inhibitors of WEEV RNA replication.

Primary HTS and validation of candidate antivirals against neurotropic alphaviruses. The pWR-LUC:BSR-T7/5 system was optimized to a 384-well HTS format and Z′-scores greater than 0.6 (Zhang et al., J Biomol Screen 1999; 4:67-73) were obtained. This optimized system was used to screen a diversity library of 51,028 compounds at the University of Michigan Center for Chemical Genomics (CCG). This composite library consisted of compounds from four smaller collections: Chembridge (13,028 compounds), ChemDiv (20,000 compounds), Maybridge (16,000 compounds), and MS Spectrum 2000 (2,000 compounds), the latter of which included FDA approved drugs. Table 1 provides a composite overview of the experimental systems, criteria, and results from the HTS and subsequent validation steps. For the primary HTS, parameters were selected to identify compounds with inhibitory activity that suppressed fLUC signal to at least 70% of the level obtained with the positive control ribavirin and obtained a hit rate of 0.4%. 82 compounds were excluded that had activity in previous LUC-based screens run at the CCG, thus reducing the selection of toxic compounds or those with direct activity against the reporter gene. The remaining 114 compounds were subjected to dose-response analysis for primary validation, where 68% of these compounds had 50% maximal inhibitory concentration (IC50) values of less than 100 μM.

New material was purchased from the original suppliers for 46 available compounds with the lowest IC50 values, and secondary validation studies were conducted with cell-based replicons derived from VEEV or EEEV that incorporated a SEAP reporter gene rather than fLUC. This step allowed for exclusion of compounds that were active against fLUC but also increased the potential of identifying compounds with broad activity against neurotropic alphaviruses. Eleven compounds showed activity in the secondary validation assays and were evaluated in tertiary validation assays with repeat detailed dose-response and toxicity assessment to calculate precise 50% cytotoxicity concentration (CC50) and IC50 values using the original pWRLUC:BSR-T7/5 system. Four compounds had toxicity:activity (T:A) ratios (CC50/IC1050) greater than 5 and were selected as candidates for further development as alphavirus inhibitors. One of these compounds, designated CCG32091, was particularly potent with a T:A ratio of greater than 20 (FIG. 2A). For comparison, ribavirin had an IC50 of 16.0 μM and T:A ratio of 19 with the pWR-LUC:BSR-T7/5 system. CCG32091, which has a thieno[3,2-b]pyrrole core

structure with a 4-fluorobenzyl R1 group attached to the pyrrole nitrogen and a 2-furanylmethylamine R2 group incorporated into the terminal carboxamide (FIG. 2B; IUPAC name 1-({4-[(4-fluorophenypmethyl]-4H-thieno[3,2-b]pyrrol-5-yl}carbonyl)-N-(furan-2-ylmethyl)piperidine-4-carboxamide), was chosen as an initial lead antiviral compound for final verification studies with live virus and structure-activity relationship (SAR) analysis.

Verification of CCG32091 antiviral activity with live virus and cultured neuronal cells. The primary target cell of neurotropic alphaviruses is the CNS neuron, and thus a final verification of the antiviral activity of CCG32091 was performed using an in vitro model with human neuronal cells previously used to study WEEV pathogenesis (Castorena et al., supra). For initial experiments with infectious virus FMV, an alphavirus closely related to WEEV, and SINV, the prototypic alphavirus used to study pathogenesis were used. Both of these viruses can be handled safely under BSL-2 conditions. One characteristic of alphavirus replication in cultured mammalian cells is the rapid development of cytopathic effect (CPE), which is due in part to virus-mediated disruption of host cell transcription and translation (Garmashova et al., J Virol 2006; 80:5686-96; Garmashova et al., J Virol 2007; 81:2472-84; Gorchakov et al., J Virol 2005; 79:9397-409 [28-30]. BE(2)-C cells were infected with FMV or SINV in the presence of 12.5 μM CCG32091 or 50 μM ribavirin and cell viability was measured at 48 h after infection by MTT assay (FIG. 3A). Treatment with CCG32091 suppressed virus-induced CPE and increased cell viability from 20% in infected but mock treated cells to 50% or 70% for SINV- or FMV-infected cells, respectively. Furthermore, CCG32091 effectively suppressed FMV-induced CPE at concentrations as low as 3 μM, the lowest concentration tested in this assay.

The ability of CCG32091 to inhibit virus replication was directed assessed by examining infectious virion production (FIG. 3B) and viral RNA replication (FIGS. 3C and D). CCG32091 suppressed infectious FMV production by >90%, similar to the level of suppression seen with the positive control ribavirin (FIG. 3B). Furthermore, when viral RNA replication was examined by RT-PCR with either WEEV- or FMV-infected BE(2)-C cells, CCG32091 reduced the accumulation of viral RNAs encoding either nsP2 or E1 by 80-90% (FIGS. 3C and D). Northern blotting confirmed that CCG32091 reduced both genomic and subgenomic RNA accumulation after infection. These results demonstrated that CCG32091 suppressed virus replication in infected neuronal cells, inhibited virus-induced CPE, and had broad activity against several alphaviruses.

SAR analysis with CCG32091. To optimize the therapeutic profile of antivirals, the structure of CCG32091 (FIG. 2B) was compared with those of compounds in the entire CCG library. Twenty compounds were identified that contained a core thieno[3,2-b]pyrrole moiety but had different combinations of R1 and R2 groups compared to CCG32091 (Table 2). Six of these compounds were previously identified as “hits” in the primary HTS and dose-response analyses for validation had already been completed (CCG32075, 32089, 32090, 32092, 32095, and 32096). Dose-response analysis of the remaining 14 compounds was performed to obtain a limited SAR for CCG32091 (Table 2). This analysis revealed an approximate 250-fold range of IC50 values, from a high of 46.8 μM to a low of 0.2 25 where 6 compounds had submicromolar IC50 values (CCG32084, 32087, 32088, 32093, 32094, and 32095). Toxicity studies were completed with these 20 compounds and it was found that 90% had CC50 values >100 μM, including 5 of the 6 compounds with submicromolar IC50 values (Table 2). At R1, there appeared to be little difference between methyl and ethyl groups (compare the 4-methylbenzylamides CCG32055 and CCG32019). However, substantially better activity was observed when the small alkyl group at R1 was replaced with 4-fluorobenzyl (compare CCG32088 with CCG32052). A direct comparison between 4-fluorobenzyl and 4-chlorobenzyl at R1 (2-furanylmethyl amides CCG32091 and CCG32095) also indicated that 4-chlorobenzyl represents a further optimization of R1. Among the amines incorporated at R2, none were clearly superior to the others. In fact, a variety of amines were seen with potent inhibitors, including 4-methylpiperidine (CCG32087), benzyl (CCG32088), isopentyl (CCG32093), and 4-(2-furanylcarboxy)piperazine (CCG32084). With regard to the internal piperidine carboxamide, the two 3-carboxamide analogs CCG32001 and CCG32009 had distinctly inferior activity compared to the closely related 4-carboxamide analogs CCG32025 and CCG32084, respectively. Overall, these results identified several additional compounds with enhanced potency but similar toxicity compared to the original compound CCG32091.

TABLE 1

| Step | Experimental system/resource | Criteria | Number of compounds |
|---|---|---|---|
| | CCG chemical diversity library | | 51,028 |
| HTS | pWR-LUC replicon and | Reduction in fLUC activity either: | 196 |
| | BSR-T7/5 cells | 1) >2 S.D. per plate from negative control; or | |
| | | 2) >90% per plate of positive control | |
| | | No activity in previous CCG LUC-based screens | 114 |
| Primary | pWR-LUC replicon and | Dose-response with IC₅₀ < 100 μM | 87 |
| validation | BSR-T7/5 cells | | |
| Secondary | EEEV/VEEV-SEAP replicon- | Dose-response with IC₅₀ < 100 μM | 11 |
| validation | bearing BHK cells | | |
| Tertiary | Repeat dose-response | Toxicity:activity ratio >5 | 4 |
| validation | with pWR-LUC replicon | | |
| | and BSR-T7/5 cells | | |

TABLE 2
CCG32091 structure-activity relationship (SAR) analysis

| Compound | | | |
|---|---|---|---|
| No. | R1 | R2 | IC₅₀ (μM) |
| 32001* | CH₃ | 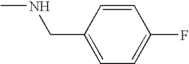 | 33.9 |
| 32009* | CH₃ | 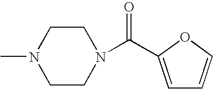 | 46.8 |
| 32052 | CH₃ | 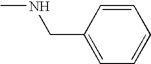 | 20.4 |
| 32055 | CH₃ | 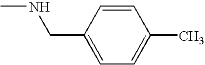 | 4.7 |
| 32075 | CH₃ | 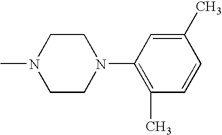 | 8.0 |
| 32084 | CH₃ | 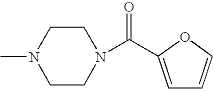 | 0.3 |
| 32019 | CH₂CH₃ | 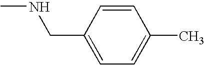 | 5.8 |
| 32023 | CH₂CH₃ | 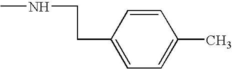 | 1.2 |
| 32025 | CH₂CH₃ | 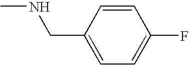 | 4.7 |
| 32044 | CH₂CH₃ | 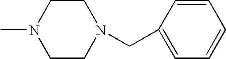 | 1.2 |
| 32048 | CH₂CH₃ | 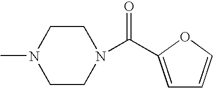 | 5.5 |
| 32087 | 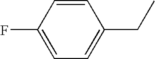 | 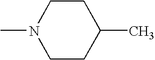 | 0.2 |
| 32088 | 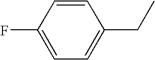 | 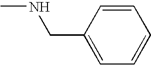 | 0.2 |
| 32089 | 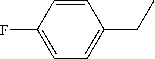 | 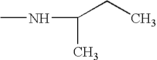 | 4.9 |
| 32090 | 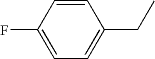 | 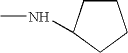 | 10.6 |
| 32091 | 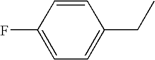 | 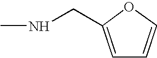 | 9.3 |
| 32092 | 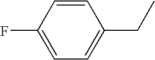 | 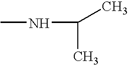 | 9.9 |
| 32093 | 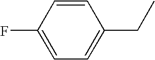 | 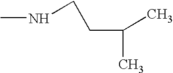 | 0.4 |
| 32094 | 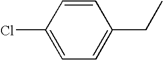 | 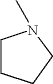 | 0.3† |
| 32095 | 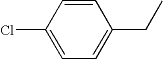 | 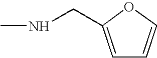 | 0.5 |
| 32096 | 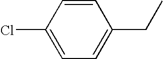 | 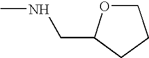 | 1.5† |

*Compounds CCG32001 and 32009 have the R2 group attached to a piperidine-3-carboxamide in contrast to a piperidine-4-carboxamide for the remaining compounds in the table.
†increased cytotoxicity compared to CCG32091 with CC₅₀ values less than 100 μM.

All publications and patents mentioned in the above specification are herein incorporated by reference. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition, comprising a compound of the formula: 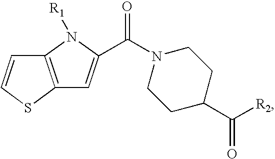
where R₁ and R₂ are the same or different and selected from the group consisting of an aliphatic group, a substituted aliphatic group, a cycloaliphatic group, a substituted cycloaliphatic group, a heterocyclic group, an aryl group, a substituted aryl group, and a halogen.

2. The composition of claim 1, wherein R₁ is selected from the group consisting of: —

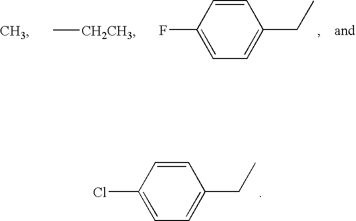

3. The composition of claim 1, wherein R₂ is selected from the group consisting of:

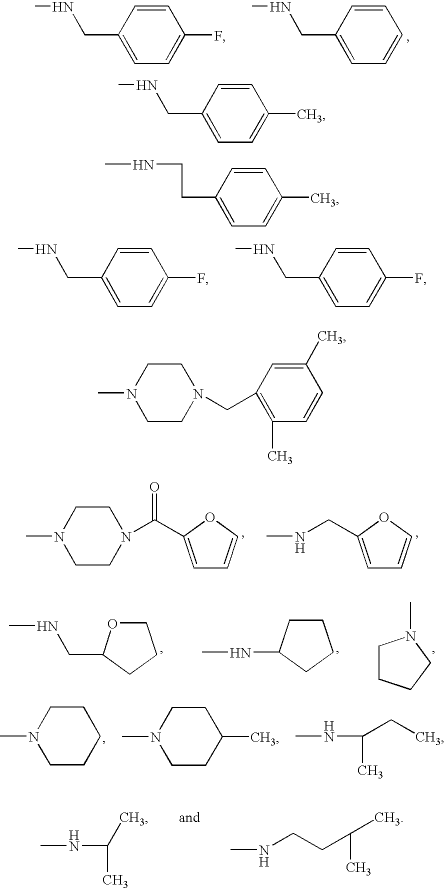

4. The composition of claim 1, wherein said compound is selected from the group consisting of

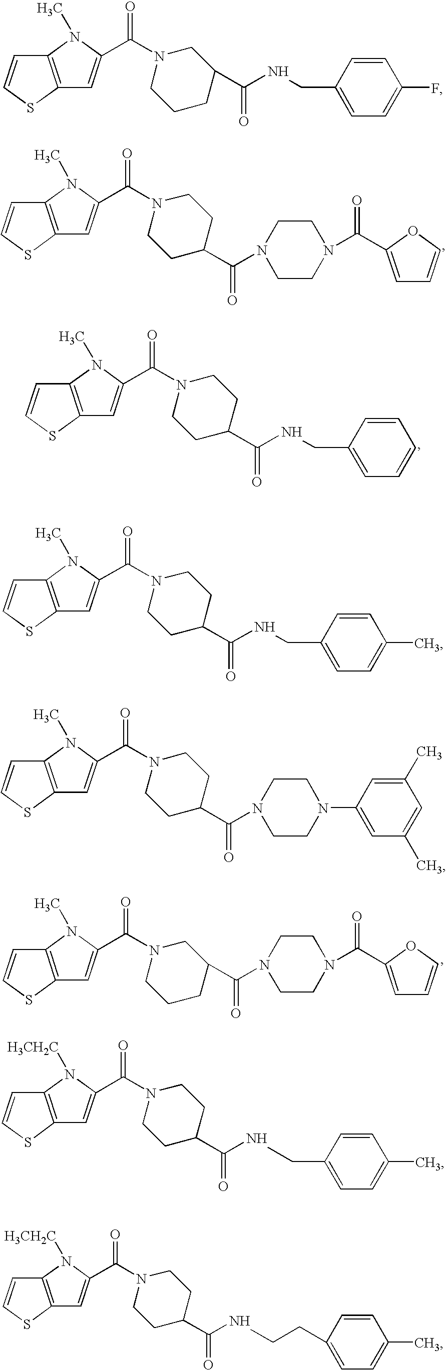

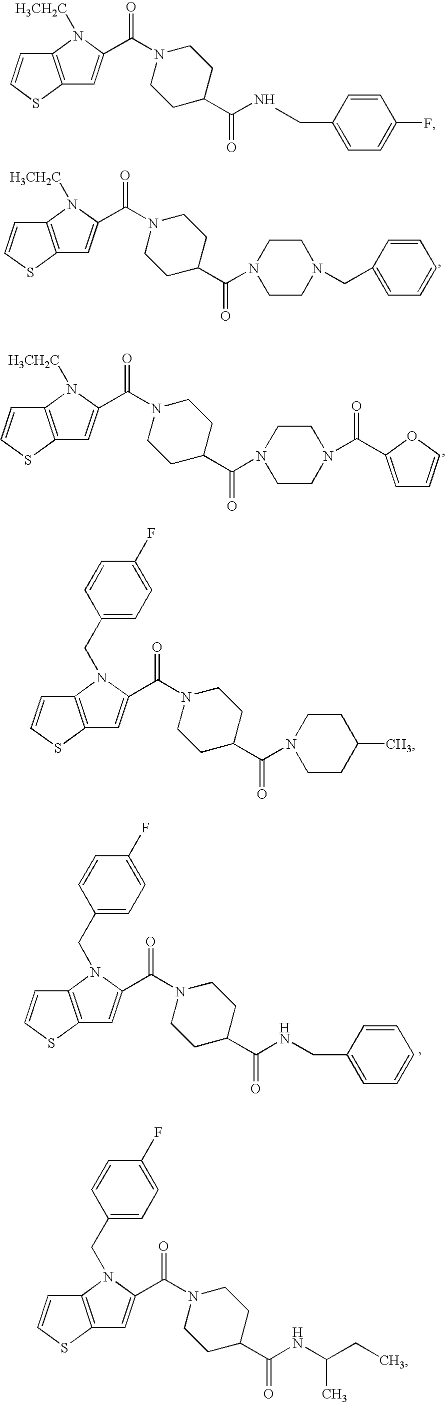

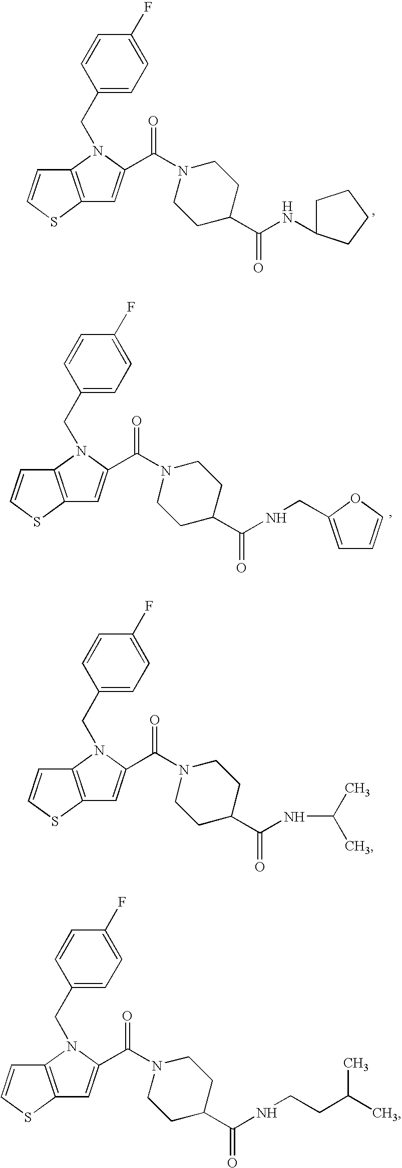

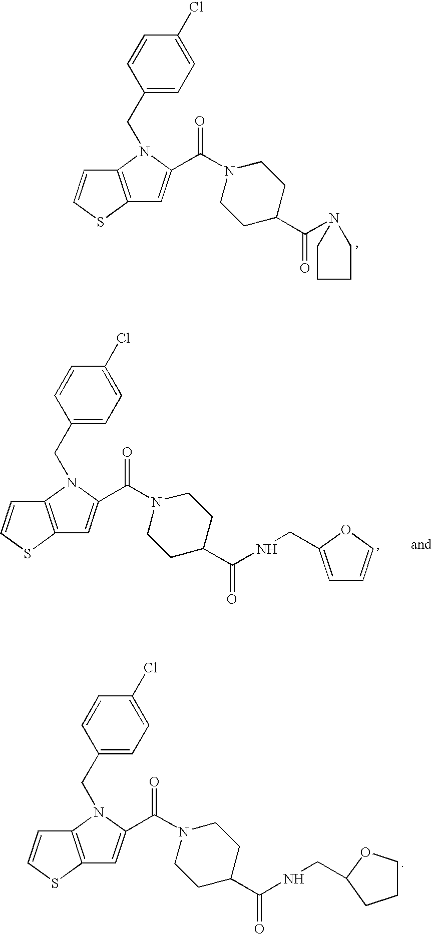

5. The composition of claim 1, wherein said compound is

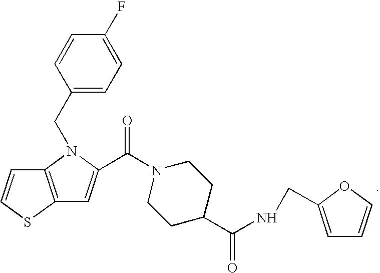

6. A pharmaceutical composition, comprising:
a) a compound of the formula 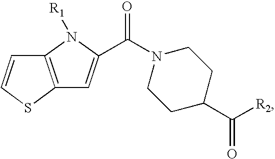
where R₁ and R₂ are the same or different and selected from the group consisting of an aliphatic group, a substituted aliphatic group, a cycloaliphatic group, a substituted cycloaliphatic group, a heterocyclic group, an aryl group, a substituted aryl group, and a halogen; and
b) a pharmaceutically acceptable carrier.

7. The composition of claim 6, wherein R₁ is selected from the group consisting of: —

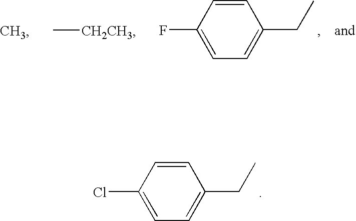

8. The composition of claim 6, wherein R₂ is selected from the group consisting of:

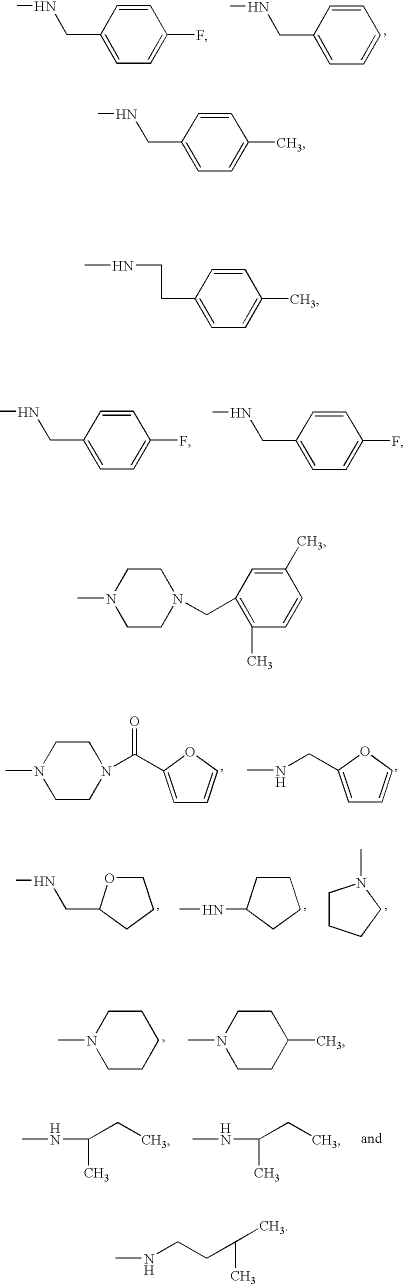

9. The composition of claim 6, wherein said compound is selected from the group consisting of

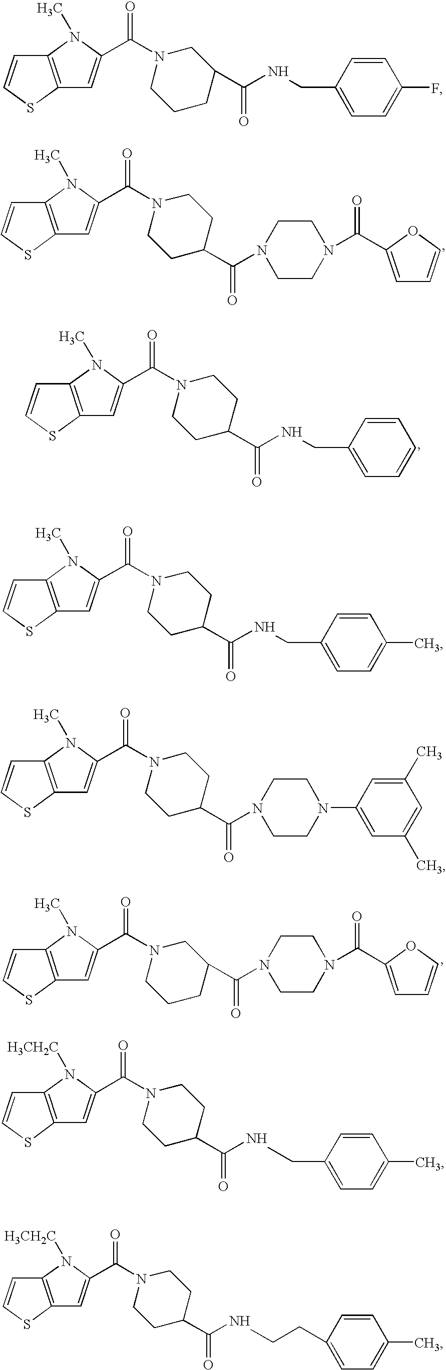

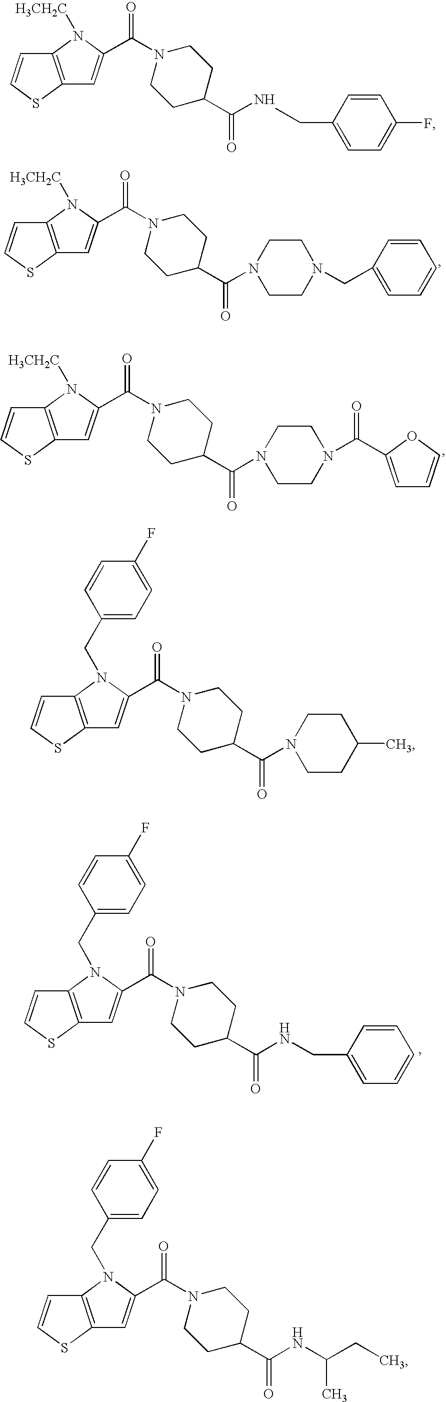

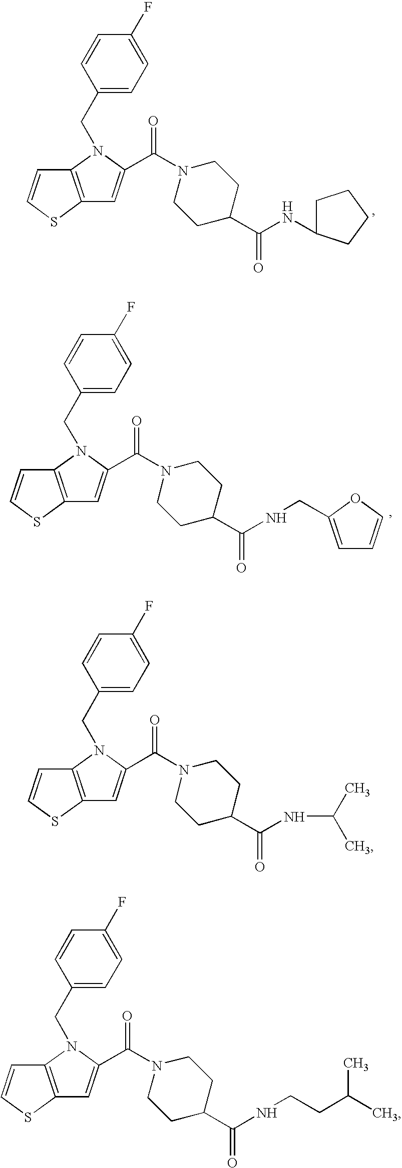

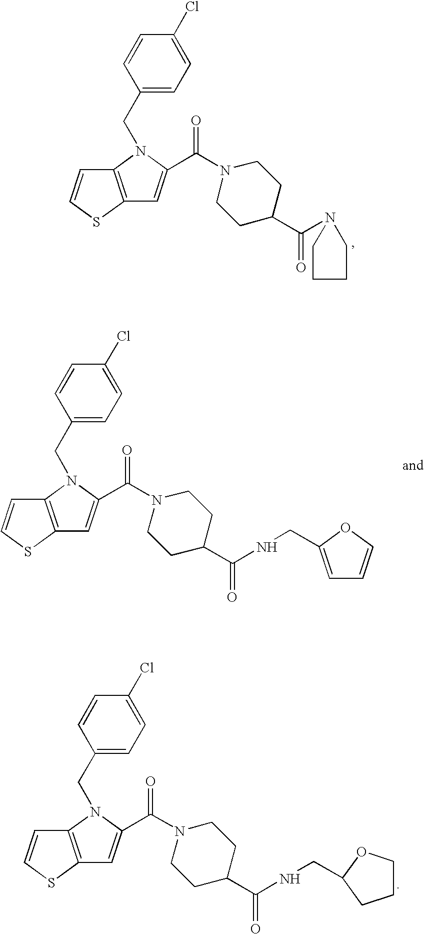

10. The composition of claim 6, wherein said compound is

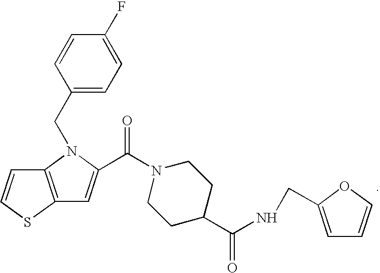

11. A method of killing or preventing the growth of an alphavirus, comprising Contacting an alphavirus with a compound of the formula 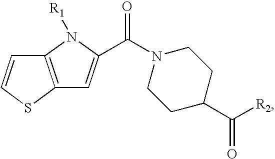
where R₁ and R₂ are the same or different and selected from the group consisting of an aliphatic group, a substituted aliphatic group, a cycloaliphatic group, a substituted cycloaliphatic group, a heterocyclic group, an aryl group, a substituted aryl group, and a halogen under conditions such that said compound kills or prevents the growth of said alphavirus.

12. The method of claim 11, wherein said alphavirus is in a cell.

13. The method of claim 12, wherein said cell is in an animal.

14. The method of claim 13, wherein said animal exhibits symptoms of an alphavirus infection and said contacting with said compound results in a decrease or elimination of said symptoms of an alphavirus infection.

15. The method of claim 13, wherein said animal is selected from the group consisting of Sindbis virus, Semliki forest virus, O'nyong'nyong virus, Chikungunya virus, Mayaro virus, Ross River virus, Barmah Forest virus, Eastern equine encephalitis virus Western equine encephalitis virus, and Venezuelan equine encephalitis virus.

16. The method of claim 11, wherein R₁ is selected from the group consisting of: —CH₃, —

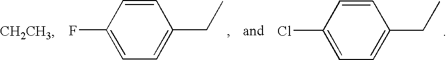

17. The method of claim 11, wherein R₂ is selected from the group consisting of:

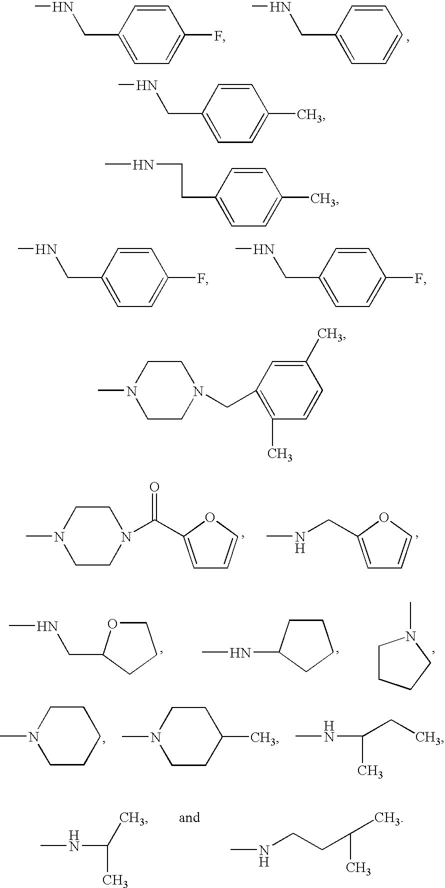

18. The method of claim 11, wherein said compound is selected from the group consisting of

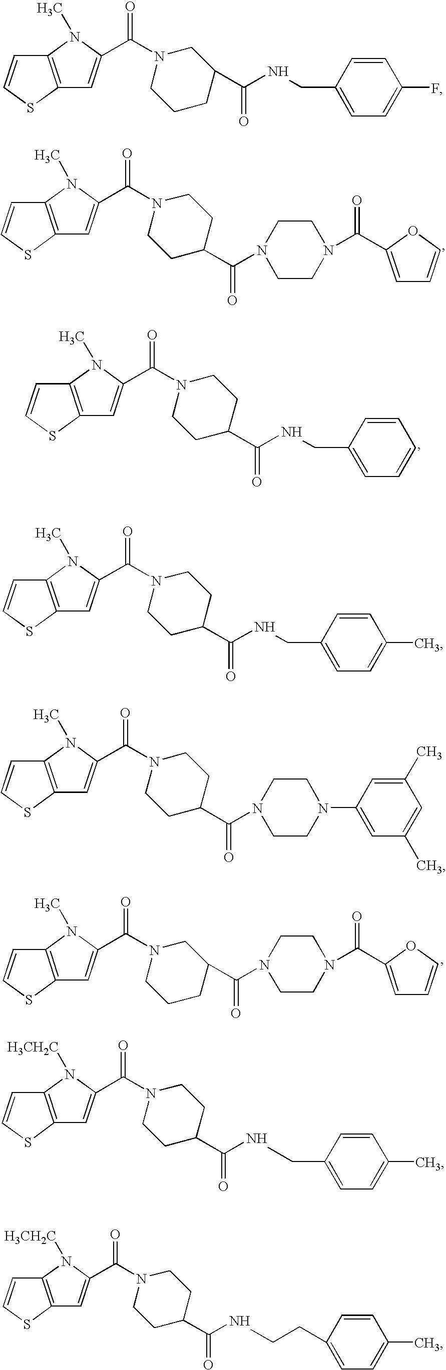

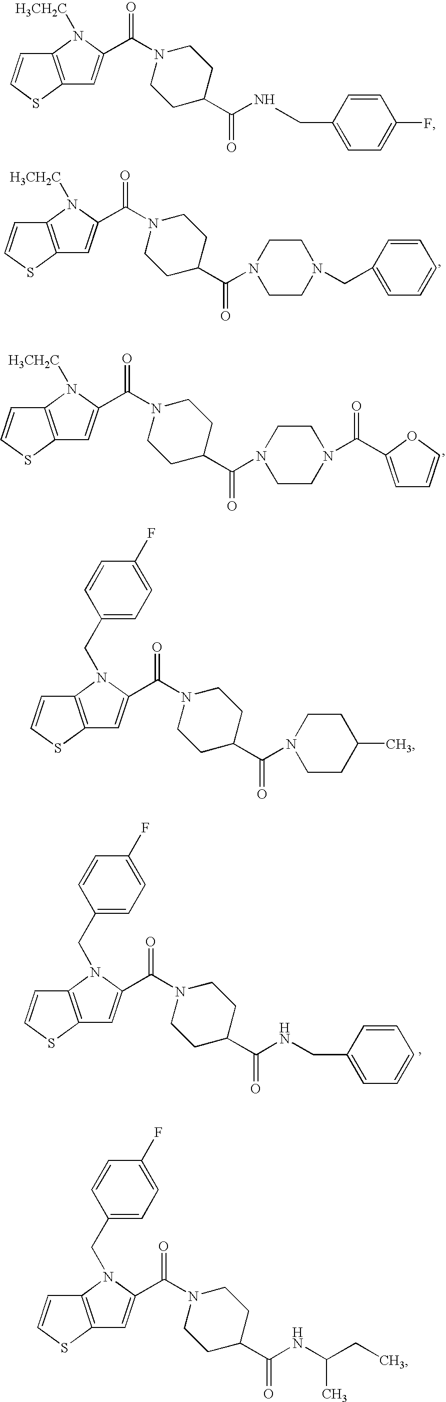

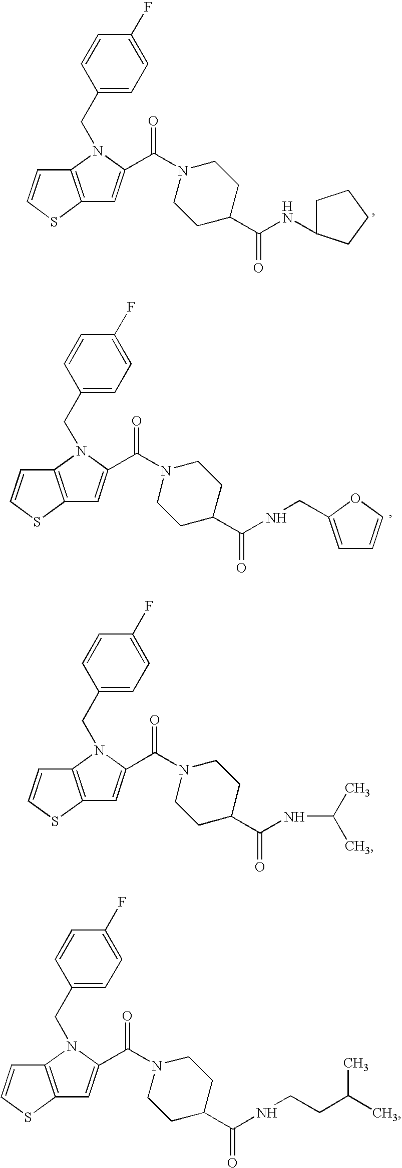

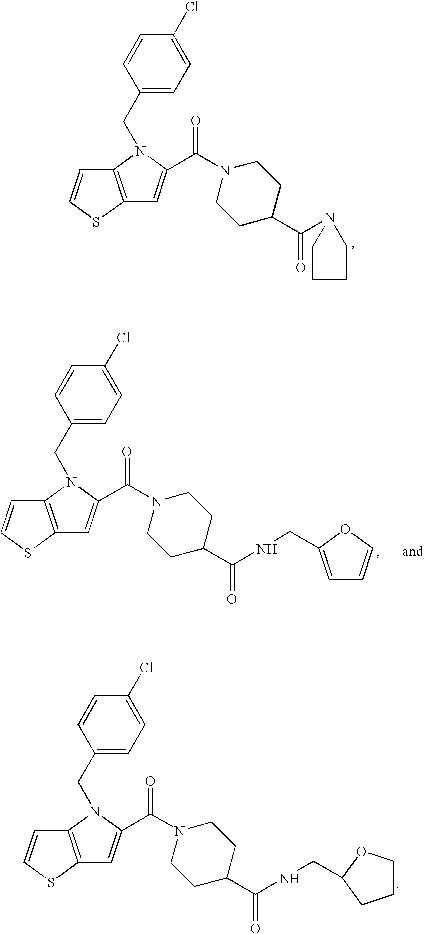

19. The method of claim 11, wherein said compound is [chemical structure]
